(19) United States
(12) Patent Application Publication
(10) Pub. No.: US 2016/0029963 A1
(43) Pub. Date: Feb. 4, 2016

(54) MEDICALLY ACTIVE TOYS

(71) Applicant: Elwha LLC, Bellevue, WA (US)

(72) Inventors: Roderick A. Hyde, Redmond, WA (US); Muriel Y. Ishikawa, Livermore, CA (US); Edward K.Y. Jung, Bellevue, WA (US); Eric C. Leuthardt, St. Louis, MO (US); Elizabeth A. Sweeney, Seattle, WA (US); Lowell L. Wood, JR., Bellevue, WA (US); Victoria Y.H. Wood, Livermore, CA (US)

(21) Appl. No.: 14/445,816

(22) Filed: Jul. 29, 2014

(51) Int. Cl. A61B 5/00; A61B 5/145; A61B 5/0205; A61B 7/00; A61B 10/02; A61B 5/0476; A61B 5/0402; A61B 5/0488; A61B 5/11; A61B 5/117; A61B 5/1455

(52) U.S. Cl. CPC A61B 5/6896; A61B 5/117; A61B 5/14517; A61B 5/02055; A61B 5/14546; A61B 7/003; A61B 5/14552; A61B 5/4875; A61B 5/0476; A61B 5/0402; A61B 5/0488; A61B 5/11; A61B 5/7475; A61B 5/0002; A61B 10/02; A61B 5/4839; A61B 5/01

(57) ABSTRACT

Embodiments are disclosed herein that relate to smart systems, methods, and devices for testing, monitoring, and/or diagnosing a subject based on assessment of one or more physiological parameters and/or biological agents. In an embodiment, a smart toy device is employed, optionally as part of a system, to engage with a subject by way of one or more sensors embedded in the toy device. In an embodiment, the toy device instructs the subject on how to engage most effectively with it in order to provide data related to the subject's disease, condition or diagnosis. In an embodiment, the toy device provides one or more rewards to the subject for complying with instructions and/or sensor engagement and/or biological testing.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)).

### PRIORITY APPLICATIONS

None.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### SUMMARY

Various embodiments disclosed herein relate to interactive medical toys for monitoring and/or diagnosing a subject through play and interaction. Various embodiments include medical toys with computer processors and/or as part of a computer system. Various embodiments disclosed include specific components for particular testing of biological tissue(s) of a subject. Various embodiments disclosed include accepting, recording, and/or transmitting data related to the subject's health to a database or other electronic record.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a partial view of an embodiment of a system with a toy device.

FIG. 2 is a partial view of an embodiment of a system with a toy device.

FIG. 3 is a partial view of an embodiment of a system with a toy device.

FIG. 4 is a partial view of an embodiment of a system with a toy device showing internal circuitry.

FIG. 5 is a partial view of an embodiment of a method employed in a system including a toy device.

FIG. 6 includes a partial view of an embodiment of components of a toy device.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

In an embodiment, the medically active toy device or interactive system includes at least one biosensor. In an embodiment, the device or system includes a plurality of biosensors, which may include a single or multiple types. In an embodiment, the device or system includes a plurality of biosensors in an array. In an embodiment, the at least one biosensor includes one or more of an optical sensor, electromagnetic sensor, magnetic sensor, electrophoretic sensor, electrochemical sensor, biochemical sensor, microelectrode sensor, chemical sensor, microfluidic sensor, magnetic resonance sensor, piezoelectric sensor, surface plasmon resonance sensor, optical microsensor array, surface enhanced raman spectrometer (SERS), laser, ion flow tube, metal oxide sensor (MOS), spectrophotometer, acoustic wave sensor, colorimetric tube, conductive- or semiconductive-polymer gas sensor, chemoresistor, selective resonance sensor, gas chromatograph, quartz microbalance sensor, optical waveguide sensor, electrochemical sensor, electrically conducting sensor, mass spectrometer, spectrophotometer, aptamer-based biosensor, ion mobility spectrometer, photo-ionization detector, amplifying fluorescent polymer sensor, ion mobility spectrometer, thickness-shear mode sensor, microgravimetric sensor, cantilever or microcantilever sensor, or carbon nanotube. For example, one or more biosensors can include a gas sensor, capillary electrophoretic device, nuclear magnetic resonance imager, an “electronic nose” or “electronic tongue.”

In an embodiment, the biosensor can include a selective detection unit, a transducing unit, and a reporter unit. For example, a reporter unit is a signal transmitter or a readout.

In an embodiment, the biosensor includes an electrochemical biosensor. For example, an electrochemical biosensor can include a recognition layer on a solid electrode surface. In an embodiment, the recognition layer may include any of a number of recognition molecules. In an embodiment, a recognition molecule may include, for example, a nucleic acid or aptamer, a protein (including an immunological protein) or peptide, or any other binding element. In an embodiment, an electrochemical biosensor may include a reaction layer on a solid electrode surface. In an embodiment, a reaction layer might include an enzyme able to bind a biological agent and in catalyzing a reaction, induce a signal. In an embodiment, an example of an electrochemical biosensor having an enzyme is a glucose sensor that utilizes glucose oxidase. In an embodiment, the electrochemical biosensor includes an electric transducer. In an embodiment, electrochemical biosensors can be fabricated in the micro- or nano-scale sizes, the latter, for example, using nanowires or nanotubes.

In an embodiment, the biosensor includes at least one quartz microbalance sensor coated with a molecular film able to bind a biological agent. In an embodiment, the biosensor includes at least one microcantilever sensor carrying at least one recognition molecule able to bind a biological agent. The binding of a biological agent to a microbalance sensor or microcantilever sensor is detected via changes in the total resonant frequency as the total mass of the sensor changes with the addition of the biological agent (see, e.g., Montuschi et al., CHEST 137(4):790-796; 2010, which is incorporated herein by reference). For example, biomarker molecules may be detected with piezoresistive microcantilever sensors that carry antibodies specific for cytokines or other biomarkers (see e.g., International Publication No. WO 2005/100235, which is incorporated herein by reference).

In an embodiment, the biosensor includes a single-walled carbon nanotube capacitance sensor carrying a selective material for detecting a specific biological agent. For example a single-walled carbon nanotube may combine a nonselective transducer with a chemoselective material that serves to concentrate and detect a volatile organic compound (see, e.g., Snow et al., Science 307 (5717):1942-1945; 2005), which is incorporated herein by reference. The carbon nanotube might utilize as its recognition site single-strand DNA (see, e.g., Staii et al., Nano Lett. 5(9):1774-1778; 2005), which is incorporated herein by reference. For example, carbon nanotube sensors may be used in a biosensor that includes, for example, an electronic nose or electronic tongue.

In an embodiment, the biosensor includes at least one bioactive matrix, such as a bioactive gel or polymer, configured with a recognition site able to bind a biological agent. In an embodiment a recognition site includes an immobilized binding molecule. In an embodiment a recognition site includes a molecularly imprinted binding site.

Molecular imprinting is described in, for example, Byrne et al., “Molecular imprinting within hydrogels,” Advanced Drug Delivery Reviews 54:149-161 (2002), Peppas and Huang, “Polymers and gels and molecular recognition agents,” Pharm Res. 19(5):578-87 (2002), and U.S. Patent Application No. 2007/0190084, each of which is incorporated herein by reference. In an embodiment, a plurality of molecularly imprinted recognition sites are included in multiple biosensors and are associated with particular locations on the device. In an embodiment, the one or more gel components are configured to recognize and respond to at least one biological agent. See, for example, Peppas and Huang, ibid. and Tanaka et al., “Polymer gels that can recognize and recover molecules,” Faraday Discuss., 101:201-206 (1995), each of which is incorporated herein by reference. For example, binding of a biological agent to the gel of a biosensor can induce changes in the matrix that alter its electrical conductance or light absorbance, which can be measured with electrodes or light source, respectively, or can induce changes in gel volume measurable by a pressure sensor or light source. For example, polymerized crystalline colloidal array (PCCA) hydrogels change volume as their biological agent is bound, inducing changes in the lattice spacing, which alter the wavelength of the diffracted light. For example, photonic PCCA hydrogels can incorporate molecules that recognize specific biological agents, for example charged molecules (e.g., that alter the gel when recognizing glucose, see, e.g., Ben-Moshe et al., Anal. Chem. 78:5149-5157, 2006 and U.S. Pat. No. 7,105,352, each of which is incorporated herein by reference), or enzymes (see, e.g., Walker et al., Anal. Chem. 77:1596-1600, 2005, which is incorporated herein by reference), or binding ligands, such as antibodies against biomarkers (see, e.g., U.S. Pat. No. 6,544,800, which is incorporated herein by reference).

In an embodiment, the medically active toy device includes at least one light source. A light source may include, for example, a light emitting diode, organic light-emitting diode, or micro light-emitting diode. A light source may include, for example, a light source configured to provide light in a variable and/or specific wavelength, including infrared or ultraviolet. See, for example, U.S. Pat. No. 5,183,740, which is incorporated herein by reference. In an embodiment, the light source is associated with the biosensor and is configured for use in optically detecting changes in the biosensor.

In an embodiment, the biosensor includes a transducer. Accordingly, the signal generated by a biosensor includes, for example, an electrical, visual, magnetic, acoustic, vibrational, heat, light (e g, infrared (IR), ultraviolet (UV), radio frequency (RF), or electromagnetic (EM) radiation signal.

In an embodiment, one or more modular components are utilized in the toy (e.g., as a cartridge that is removable, disposable, or interchangeable for a cartridge of another kind—such as for analyzing different substances). In an embodiment, one or more biological fluids include at least one of a bodily liquid or a gas, such as an exhaled or eliminated condensate or gas.

In an embodiment, the biosensor includes a detector for detecting a biomarker. In an embodiment, the biomarker includes at least one metabolite of a pathogen. For example, metabolites of H. pylori include urea and ammonia (see, e.g., Marais et al., Microbiol. Mol. Biol. Rev. 63(3):642-674, 1999, which is incorporated herein by reference).

In an embodiment, the biomarker includes at least one volatile organic compound. In an embodiment, the volatile organic compound includes at least one metabolite of a pathogen. For example, metabolites of M. tuberculosis include oxetane, 3-(1-methylethyl)-, dodecane, 4-methyl-, cyclohexane, hexyl-, bis-(3,5,5-trimethylhexyl)phthalate, benzene 1,3,5-trimethyl-, decane, 3,7-dimethyl-, tridecane, 1-nonene, 4,6,8-trimethyl-, heptane, 5-ethyl-2-methyl-, 1-hexane, 4-methyl-, 1,3,5-trimethylbenzene, or 1,2,3,4-tetramethylbenzene. See, for example, Phillips, et al., Tuberculosis; 26 Jan. 2010; pp. 1-7, which is incorporated herein by reference.

In an embodiment, the device or system includes at least one of a power source, antenna, or display. The power source can include, for example, a battery, a thin film battery, a rechargeable battery, a fuel cell, or a solar cell.

In an embodiment, the subject is a mammal, reptile, bird, fish, or amphibian. In an embodiment, the subject is a human. In an embodiment, the subject is a child, disabled person, convalescent person, elderly person, or infant. In an embodiment, the subject is dog, cat, hamster, guinea pig, rabbit, or other pet. In an embodiment, the subject is afflicted with at least one disease or disorder. In an embodiment, the subject is afflicted with a chronic disease or disorder.

In an embodiment, the medically active toy can be programmed to engage with the subject in any of a number of verbal languages, or in a haptic, auditory, or other manner of communication.

In an embodiment, the subject is afflicted with at least one of diabetes, cancer, epilepsy, Crohn's disease, arthritis, pneumonia, asthma, allergies, heart disease, pulmonary disease, or chronic inflammation.

In an embodiment, the medically active toy device includes means for collecting at least one biological sample from a subject. In an embodiment, a sampling apparatus of the device may be directly coupled to the biosensor. In an embodiment, the medically active toy device is configured to sample at least one of saliva, mucus, tears, perspiration, blood, skin, skin, hair, or other biological fluid/tissue of the subject using the toy. In an embodiment, the medically active toy device is configured to sample at least one gas, e.g., an exhaled gas, from the subject's body. In an embodiment, the medically active toy device includes means to collect a sample from a bodily tissue, e.g., from an interstitial space, using minimally invasive means, including transdermal sampling. In an embodiment, a transdermal sampling apparatus of the device may employ one or more of iontophoresis, microdialysis, electromagnetic, osmosis, electroosmosis, sonophoresis, phonophoresis, magnetophoresis, suction, electroporation, microthermal ablation, microporation, photomechanical wave, microneedle, microfine cannula, microneedles, or skin permeabilization. In an embodiment, the sample includes DNA, protein, mRNA, or a pathogen.

In an embodiment, the medically active toy includes a disposable component or a re-usable component such as a component that is able to be sterilized. For example, a disposable cover or wrapping on one or more components of the toy that are used for biological tissue sampling of the subject. In an embodiment, the disposable component includes a single-use or multi-use with same subject component.

In an embodiment, the medically active toy takes the form of a doll, animal, vehicle (e.g., truck, car, etc.), sports equipment, or other toys. In an embodiment, the medically active toy takes the form of a handheld game (e.g., video game). In an embodiment, the medically active toy includes a computer processor and optionally computer memory that is configured for engaging with the subject. For example, the toy can instruct the subject to hold it in a specific manner or position (e.g., in order to better sample a biological tissue), or to hold still for a specific amount of time (e.g., in order to ensure an accurate testing of a biological tissue), or to inhale/exhale, walk or change position, hug the toy, squeeze the toy, touch a specific component of the toy, etc. In an embodiment, the medically active toy includes a biosensor associated with an aspect of the toy engageable by the subject. For example, a biosensor for testing sweat components that uses a chemical sensor may be included in the tongue of a toy animal. For example, a biosensor for testing sweat components that uses a chemical sensor may be included in the handle of a handheld game. For example a biosensor for testing exhaled gas that uses an electronic nose may be associated with the nose or ear of a toy animal. For example, a biosensor for testing a transdermal sample may be included in the hand of a toy doll. For example a nonconductive electrooculogram sensor may be included in the eyepiece of a picture viewer similar to that of a ViewMaster or in toy binoculars.

In an embodiment, the medically active toy with the computer processor engages with the subject in order to query the subject regarding physical or mental health symptoms or condition. For example, the toy can ask the subject how it is feeling today, if it has any pain, numbness, nausea, fatigue, or what the emotional state of the subject is, if it is happy or sad, lonely or depressed, etc. In an embodiment, the medically active toy includes a computer processor and corresponding computer programs or applications that have been programmed to respond according to the answers given by the subject. For example, if the subject states that it has pain, the toy can ask, “Where does it hurt?” In an embodiment, the medically active toy simultaneously or sequentially tests the subject (e.g., breath, perspiration, heart rate, blood pressure, pupil diameter, etc.) in order to assess the subject's health status and verify the subject's location and/or severity of self-disclosed symptom(s).

In an embodiment, the medically active toy interacts with the subject in a manner that allows the subject to ask the toy questions. For example, the subject may ask if it can take, for example, ibuprofen, without having a drug interaction with another pharmaceutical. In an embodiment, the medically active toy is configured to be able to access the subject's electronic health records and/or other medical databases. In an embodiment, the data analysis of the medically active toy can include a determination based on other medical information, either stored in memory, accessed by way of a database or electronic record, or based on entered information from the subject. In an embodiment, the data can be transmitted by way of a conduit, wire, network, or other transmission mode.

In an embodiment, the medically active toy interacts with the subject in such a manner as to diagnose a disease or condition. For example, if the subject is coughing or has tremors that had previously been undiagnosed, the medically active toy can record the data, optionally transmit it to a subject, or second or third party (a healthcare worker, an electronic medical record, or a computer). In an embodiment, the medically active toy includes a computer processor with computer programs including algorithms that can be utilized for comparison and determination of potential diagnosis based on the symptoms recorded/transmitted or self-reported by the subject (e.g., through questioning the subject). In an embodiment, the medically active toy is configured to likewise propose adjusting a drug treatment plan or other course of treatment relating to the subject's physical or mental health.

In an embodiment, the medically active toy requires a log in or password in order to be activated and engage with the subject, and in order to ensure that the toy is engaging with the appropriate subject or to collect information identifying the subject. In an embodiment, the log in or password is entered through an input device. In an embodiment the medically active toy includes means for collecting data for use in identification. In an embodiment, the toy includes an RFID scanner (for scanning an RFID tag on the subject), optical scanner or imager, acoustic scanner, or other biometric data scanner for capturing data associated with the subject. For example, data may include a verbal word, or sound (e.g., for voice recognition), or fingerprint or eye (e.g., iris or retina) pattern, or other form of log in, including but not limited to a unique identifier (e.g., DNA, microbiome profile of a subject's skin or saliva, heart beat pattern, brain wave pattern, etc.) of the subject. For example, various physiological and/or biochemical attributes or measurements can be used.

In an embodiment, the medically active toy has means to positively identify the appropriate subject of the medical toy device. In an embodiment, the various physiological and/or biochemical attributes or measurements are combined for an even higher level of accuracy of identification of the subject. As disclosed herein, identifying information includes one or more of a saliva test (for example, for determining bacteria populations that include bacteria unique to a subject), heart beat pattern, or brain wave pattern (for example, as measured by a brain wave reading headset or a screen or toy that is controlled by the subject's unique brain waves, for example using a nonconductive remote EEG. In an embodiment, the subject is confirmed to be the appropriate subject based on facial recognition, or one or more security questions presented to the subject to verify its identity. In an embodiment, the medically active toy device includes an optical scanner that reads a barcode or other code from a subject. For example, the subject wears a wristband, ring, pendant, necklace, or other accessory with a unique barcode or other encoded identification. The medically active toy includes an optical scanner embedded or placed on it that reads the barcode or other encoded information when scanned (e.g., by the subject contacting a matching depression or pattern on the toy—a “lock and key” configuration or matching symbol or pattern).

In an embodiment, verification of the subject's identification is conducted by the medically active toy prior to engaging with the subject on any level. In an embodiment, verification of the subject's identification is conducted at random intervals once the medically active toy has been engaged with the subject using it. In an embodiment, verification of the subject's identification is conducted at regular intervals or each time a measurement is taken by the toy (e.g., a sensor engages, a needle samples, etc.).

In an embodiment, a medically active toy accessible to more than one subject includes means (described herein) to establish identification of the subject using the toy. In an embodiment information regarding the identification of the subject is encoded in the sensor signal. In an embodiment, circuitry and programming process information regarding the identification. In an embodiment, information regarding the identification of the subject is included in transmitted information.

In an embodiment, a reward mechanism is built into the feedback loop of the toy engaging in interaction with the subject. For example, in an embodiment when a blood sample is required, the toy may instruct the subject to hold it or place it on a particular location of the subject's body, and once the blood sample is drawn the toy may generate an audio/visual reward (graphic of cartoon dancing or celebrating, lights flashing or ‘jackpot’ visual, or colors or patterns to indicate a success, etc.). In another example, points may be earned by complying with the blood sample (or receipt of medication, etc.) that may be used for discounts, prizes, etc. In an example, a coin, game piece, money, or other reward is utilized for biological sampling or for therapeutic or nutraceutical receipt by the subject.

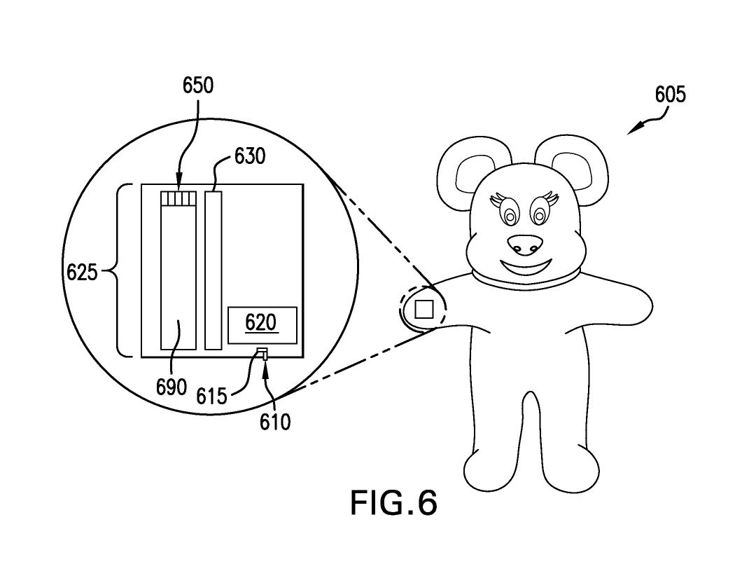

In an embodiment, a label is included in a separate compartment of the device, and the label is released in response to a specific time, or in response to detection of a specific biological agent. See FIG. 6 for details. In an embodiment, the label can include a dye, luminescent substance, fluorescent substance, magnetic compound, or quantum dot. In an embodiment, the label is included in a matrix that is released when a specific biological agent binds, such as in a displacement assay.

In an embodiment, at least one compartment of the device includes a storage container for one or more therapeutic agents or nutraceutical agents (e.g., an aromatherapy agent, vitamin, mineral, herbal supplement, or other nutraceutical agent) to be administered or provided to the subject of the medical toy. See FIG. 6 for details. In an embodiment, the storage container is a bottle, bin, membrane, or similar holding container. In an embodiment, the storage container is configured with a gate, door, recess, spring, opening, nozzle, or other structure that allows for dispensing of the therapeutic agent or nutraceutical agent to the subject. As described herein, many modes of administering one or more therapeutic or nutraceutical agents can be utilized with the device (e.g., gas, mist, topical, injection, etc.). For example, a reservoir that includes one or more therapeutic or nutraceutical agents can include a pump or gel or squeezable component that exudes the topical therapeutic or nutraceutical agent to the subject. See Figures regarding therapeutic or nutraceutical agent delivery reservoir. In an embodiment, the therapeutic agent or nutraceutical agent is delivered by way of inhalable extract or spray to the subject. As described herein, the therapeutic or nutraceutical reservoir includes a mister or nozzle for dispensing the therapeutic or nutraceutical agent to the subject for inhalation delivery.

In an embodiment, the at least one therapeutic agent includes at least one of an anti-inflammatory agent, an antimicrobial agent, a chemotherapeutic agent, respiratory therapy, or a diabetes treatment agent. Non-limiting examples of an antimicrobial agent include an antibiotic, antifungal agent, or antiviral agent. In an embodiment the at least one therapeutic agent includes at least one hormone, e.g., a growth hormone or corticosteroid. Non-limiting examples of a respiratory agent include a corticosteroid, a bronchodilator, a beta-agonist, an antihistamine, a cytokine or leukotriene modifier, or a biologic. In an aspect, the at least one diabetes treatment agent includes a form of insulin. Non-limiting examples of insulin include rapid acting insulin, short-acting insulins, intermediate-acting insulins, premixed insulins, or long-acting insulins. Commercial sources of insulin are available from, e.g., Eli Lilly (Indianapolis, Ind.), Sanofi-Aventis (Bridgewater N.J.), Novo Nordisk Inc. (Princeton, N.J.), or Pfizer (New York, N.Y.).

For example, in an embodiment, a nutraceutical agent is released through a gel, mist or spray, vapor, or other aromatherapeutic delivery mode to reduce anxiety, improve sleep, increase focus, increase appetite, reduce nausea, increase memory, slow heart rate or assist in regulating breathing, etc. Examples of nutraceutical agents include, but are not limited to, lavender (sleep aid, stress relief, etc.), ylang-ylang (sleep aid, stress relief, etc.), chamomile (sleep aid, stress relief, etc.), rose (stress relief, etc.), citrus (stress relief, increased focus, etc.), cocoa absolute (stress relief, increase appetite, etc.

In an embodiment, the medically active toy includes a biosensor able to sense a biomarker in exhaled gas that indicates an asthma attack may be occurring and may further include an oxygen sensor, which together sense onset of a respiratory condition (e.g., oxygen intake levels or carbon dioxide output levels are not within an acceptable range) and deploys release of a therapeutic agent (e.g., flovent, albuterol, etc.) or nutraceutical agent in the vicinity of a subject's nose/mouth. For example, if the subject were playing and experienced an asthma attack, the medically active toy, upon sensing a potentially compromised airway situation, can release anti-inflammatory agents such as flovent, or a bronchodilator such as albuterol, or can inject cortisol or epinephrine, etc. to reduce the symptoms and alleviate a potential crisis. In an embodiment, the medically active toy further is activated to sound an emergency alarm, either to the subject directly (e.g., audio, visual, or haptic cues), and/or to a third party (e.g., transmission of a distress call to a healthcare worker or caretaker), for example as an electronic buzzer or notification on the third party's cell phone or other electronic device, or a warning system by internet or other computer system or network.

In an embodiment, the medically active toy follows a pre-determined or adaptable program including monitoring the subject, treating the subject, sensing the subject, treating the subject, sensing the subject, etc. in a feedback loop.

In an embodiment, the medically active toy includes only therapeutic agents. In an embodiment, the medically active toy includes only nutraceutical agents. In an embodiment, the medically active toy includes both therapeutic agents as well as nutraceutical agents.

In an embodiment, the medically active toy includes one or more biosensors for detecting biomarkers related to anxiety, autism, self-harm symptoms, irregular breathing (e.g., Sudden Infant Death Syndrome), or other physiological attributes or parameters. In an embodiment, the medically active toy senses a particular physiological attribute (e.g., heartbeat patterns, breathing patterns, brain wave patterns, etc.) and in an embodiment, the medically active toy further senses one or more parameters of a particular physiological or biochemical attribute (e.g., heart rate, breathing rate, brain wave rate, body temperature, blood sugar level, infection, or other specific measurement of one or more attributes of the subject).

In an embodiment, a confirmation of receiving the therapeutic or nutraceutical agent and/or taking it is required before the reward is provided to the subject. For example, a camera embedded in the toy records the subject taking the therapeutic or nutraceutical agent that is dispensed from the storage container. In an embodiment, once the camera records the subject taking (or self-administering, or having the therapeutic or nutraceutical agent administered by the toy) the therapeutic or nutraceutical agent, the toy rewards the subject as disclosed herein. In an embodiment, the storage container is disposable, replaceable, or removable.

In an embodiment, the toy includes an alarm or other immediate indication system if the subject is detected to have a serious medical condition (e.g., loss of consciousness, no breathing detected, no heartbeat detected, etc.) or if a specific medic alert is programmed into the toy (e.g., allergy, seizure risk, etc.) and the subject has at least one detected biological agent or behavior that is believed to be associated with the specific medic alert.

In an embodiment, a hydrogel or other matrix structure is utilized for encapsulated materials (e.g., slow release materials, responsive release materials, etc.), which can be labeled for detection of their degradation or use. In an embodiment, the encapsulated material can be used in the toy for detecting a biological agent from the subject. In an embodiment, the encapsulated material is a therapeutic or nutraceutical agent for the subject. In an embodiment, the encapsulated material is part of the biosensor (e.g. a responsive gel operably coupled to a transducer that converts the response of the gel into a signal). In an embodiment, the encapsulation itself may regulate the sensor, for example, as a slow-degradation of encapsulation materials.

A hydrogel may be constructed for either slow release or responsive release as desired in a particular embodiment. In an embodiment, one or more hydrogels may be retained in a reservoir within the device. In an embodiment, reservoir containing a hydrogel may be configured to actively or passively release a therapeutic or nutraceutical agent. For example, a reservoir may include a slow-release gel.

In an embodiment, the medically active toy includes one or more microfluidic components. For example, microfluidic components can include means to collect, cool, or analyze a sample from the subject. For example, breath condensate can be collected by microfluidic components that are constructed as a cassette which can be inserted in the toy bear and removed as needed. For example, microfluidic systems to detect proteins, antigens, lipids and small molecules are described (see e.g., Fan et al., Nat. Biotechnol. 26:1373-1378, 2008 and U.S. Patent App. Pub. No. 2010/0285082; each of which is incorporated herein by reference

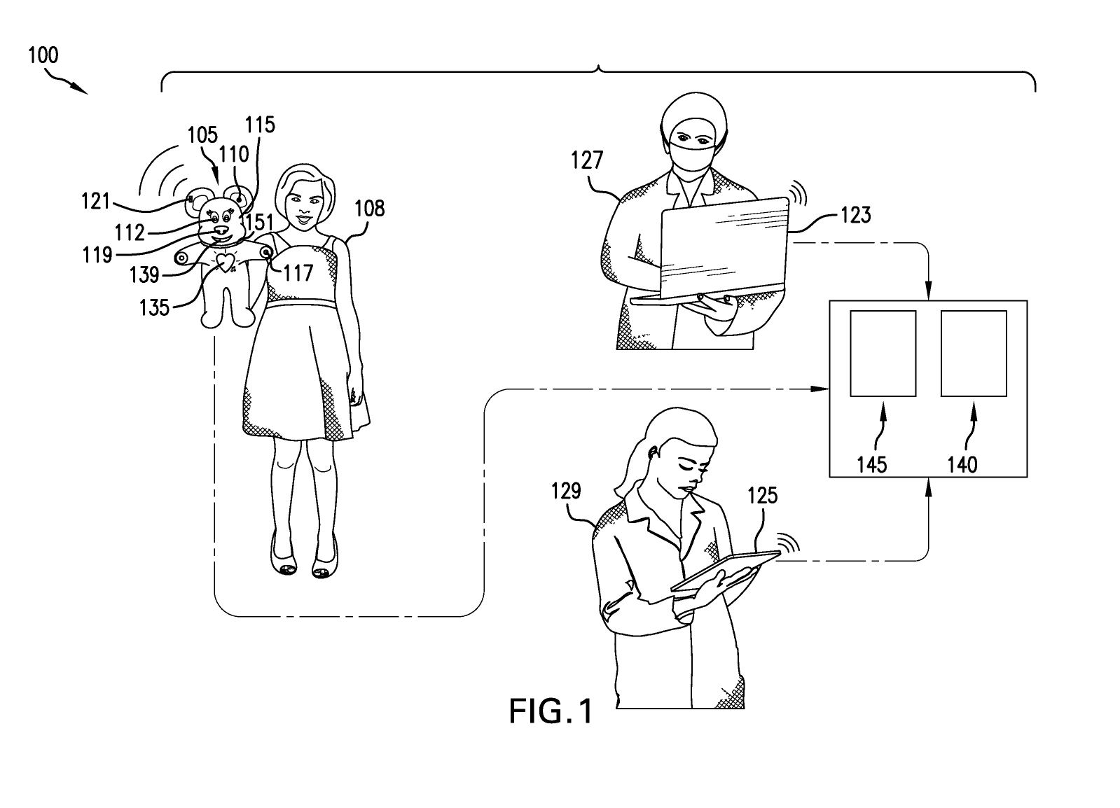

As shown in FIG. 1, in an embodiment, the system 100 includes a toy device 105 that can be handheld or otherwise physically contactable with the subject, and may include any appropriate component for interfacing with a subject 108 as described herein. In an embodiment, the medically active toy device 105 may include hardware, or a combination of hardware and software. In an embodiment, the toy is able to detect whether it is physically contacting the subject sufficiently (e.g., by way of a thermal sensor or moisture sensor) for accurate readings of other sensors or other detection modes (e.g., engaging in conversational exchange with the subject). In an embodiment, the medically active toy is not required to be physically contacting the subject (e.g., for detecting breathing or oxygen levels in cases of assessing asthma or anxiety or to use nonconductive remote sensors for EEG, EKG, etc.) in order for it to accurately measure one or more physiological or biochemical attribute or parameter.

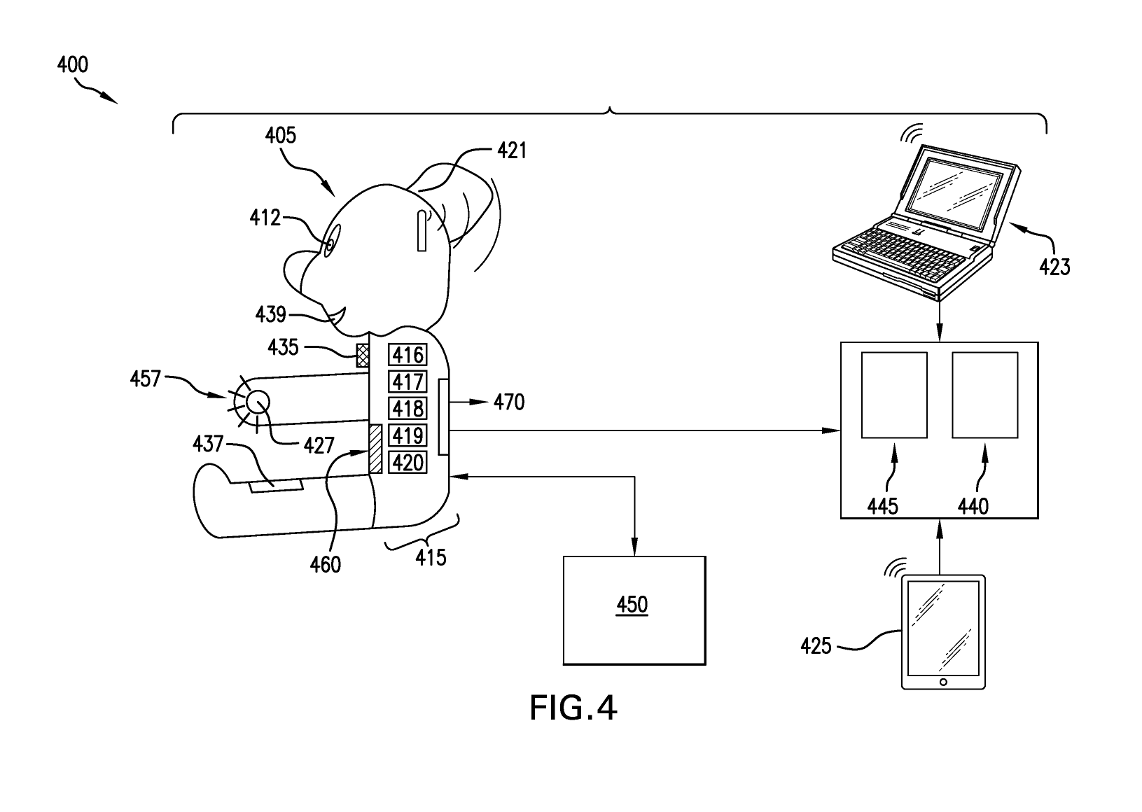

In an embodiment, an audio recorder or microphone 110 and/or a video recorder or camera 112 are included as additional sensors in the device 105. A processor 115, and the corresponding circuitry in operable communication with the sensors of the device is described in detail in FIG. 4.

In an embodiment, one or more sensors (thermal, moisture, etc.) 117 are provided in other areas of the toy device (e.g., the hand/paw of the toy). In an embodiment, a biosensor, such as an analytical sensor (e.g., electronic nose) 119 is included as a sensor, e.g., for detecting an eliminated gas such as exhaled nitric oxide or volatile organic compounds, etc. In an embodiment, the biosensor includes, for example, a chemical sensor, gas sensor, or nucleic acid sensor. In an embodiment, data from the one or more sensors is communicated by way of one or more 121 of a transmitter, receiver, or transceiver configured to communicate with a network, server, remote computing device 123, mobile device 125 (phone, tablet, etc.) that is accessible to a healthcare worker 127 or parent/guardian or caregiver 129, and the system 100 may interact with an electronic health database 145 or subject's electronic health record(s) 140. In an embodiment, a reward mechanism 135 is included (e.g., flashing lights, musical songs, words of congratulations, etc.) in response to a subject engaging with the device 105 based on the activation of one or more sensors of the device 105. In an embodiment, the device 105 instructs the subject 108 to generally or specifically engage with one or more sensors 117 of the device 105. In an embodiment, the sensors 117 randomly engaged by the subject 108 without prompting are utilized to collect data. In an embodiment, an alert mechanism 137 is included that warns of detection of abnormal data (e.g., biological agents or physiological parameters, etc.) Non-limiting examples of an alert mechanism include a red light on the collar or another part of the toy, a buzzing noise, siren, or other warning sound, or sounds, e.g., from the toy's mouth. In an embodiment, a voice recorder or digital voice 139 is provided and may be programmed to communicate with a subject or for real-time conversing with the subject by way of remote control by a healthcare worker or caregiver. The voice recorder or digital voice 139 may instruct the subject 108 to burp the baby doll, hold the toy, hold the baby doll's hand, etc. in order to better engage the subject with the sensors in different particular areas of the toy.

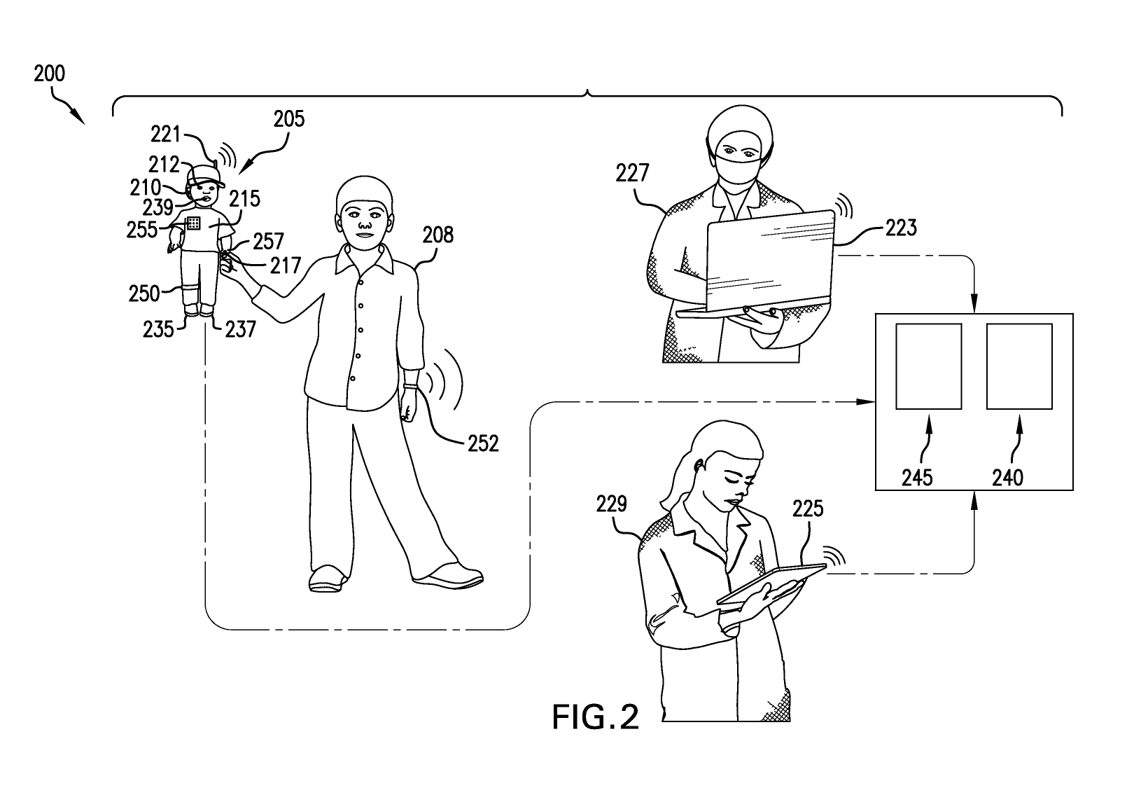

In FIG. 2, the system 200 includes a medically active toy device 205, and may include any appropriate component for interfacing with a subject 208 as described herein. In an embodiment, the device 205 may include hardware, or a combination of hardware and software, including, for example, facial or voice recognition software. In an embodiment, the device 205 is handheld or otherwise physically contactable with the subject.

In an embodiment, an audio recorder or microphone 210 and/or a video recorder or camera 212 are included as sensors in the device 205. A processor 215, and the corresponding circuitry in operable communication with the sensors of the device is described in detail in FIG. 4.

In an embodiment, one or more additional sensors (thermal, moisture, etc.) 217 are provided in other areas of the toy device (e.g., the hand/paw of the toy). In an embodiment, the medically active toy requires a log in or password in order to be activated and engage with the subject, and in order to ensure that the toy is engaging with the appropriate subject or to collect information identifying the subject. In an embodiment, the log in or password is entered through an input device. In an embodiment the medically active toy includes means for collecting data for use in identification. In an embodiment, the toy includes an RFID scanner (for scanning an RFID tag on the subject), optical scanner or imager, acoustic scanner, or other biometric data scanner for capturing data associated with the subject. In an embodiment, data from the one or more sensors is communicated by way of one or more 221 of a transmitter, receiver, or transceiver configured to communicate with a network, server, remote computing device 223, or mobile device 225 (phone, tablet, etc.) that is accessible to a healthcare worker 227 or parent/guardian or caregiver 229, and the system 200 may interact with an electronic health database 245 or subject's electronic health record(s) 240. In an embodiment, an optical scanner (not shown) is included on the device for scanning the patient's face, eye, or identification tag (e.g., a barcode on a coordinate accessory worn by the user such as a wristband, ring, necklace, pendant, etc.) for use in positive identification of the subject. In an embodiment, the toy includes an emergency alarm button or lever (not shown) that may be activated by the subject using the device if the subject has heightened symptoms (e.g., asthma attack, anxiety attack, heart palpitations, etc.) or the user needs immediate medical intervention.

In an embodiment, a reward mechanism 235 is included (e.g., flashing lights, musical songs, words of congratulations, etc.) in response to a subject engaging with the device 205 based on the activation of one or more sensors of the device 205. In an embodiment, the device 205 instructs the subject 208 to generally or specifically engage with one or more sensors 217 of the device 205. In an embodiment, the sensors 217 randomly engaged by the subject 208 without prompting are utilized to collect data. In an embodiment, an alert mechanism 237 is included that warns of detection of abnormal data (e.g., biological agents or physiological parameters, etc.). Non-limiting examples of an alert mechanism include a red light on the collar or another part of the toy, a buzzing noise, siren, or other warning sound, or sounds from the toy's mouth. In an embodiment, a voice recorder or digital voice 239 is provided and may be programmed to communicate with a subject or for real-time conversing with the subject by way of remote control by a healthcare worker or caregiver. The voice recorder or digital voice 239 may instruct the subject 208 to burp the baby doll, hold the toy, hold the baby doll's hand, etc. in order to better engage the subject with the sensors in different particular areas of the toy.

In an embodiment, one or more transdermal sampling means 255, e.g. one or more first sets of microneedles, are utilized to access tissues of the subject 208 and provide a sample for the biosensor to test (e.g., for glucose, cholesterol antibodies, or other biological agent). In an embodiment, one or more transdermal delivery means 257, e.g., one or more second sets of microneedles or one or more iontophoretic delivery apparatus, are utilized for administering a therapeutic or nutraceutical agent (e.g., insulin). In an embodiment, part of the system 200 relates to the subject 208 wearing a data recording bracelet 252, that may transmit or receive information (e.g., identification information, data derived from the subject's interaction with the toy device, or medical history data, etc.).

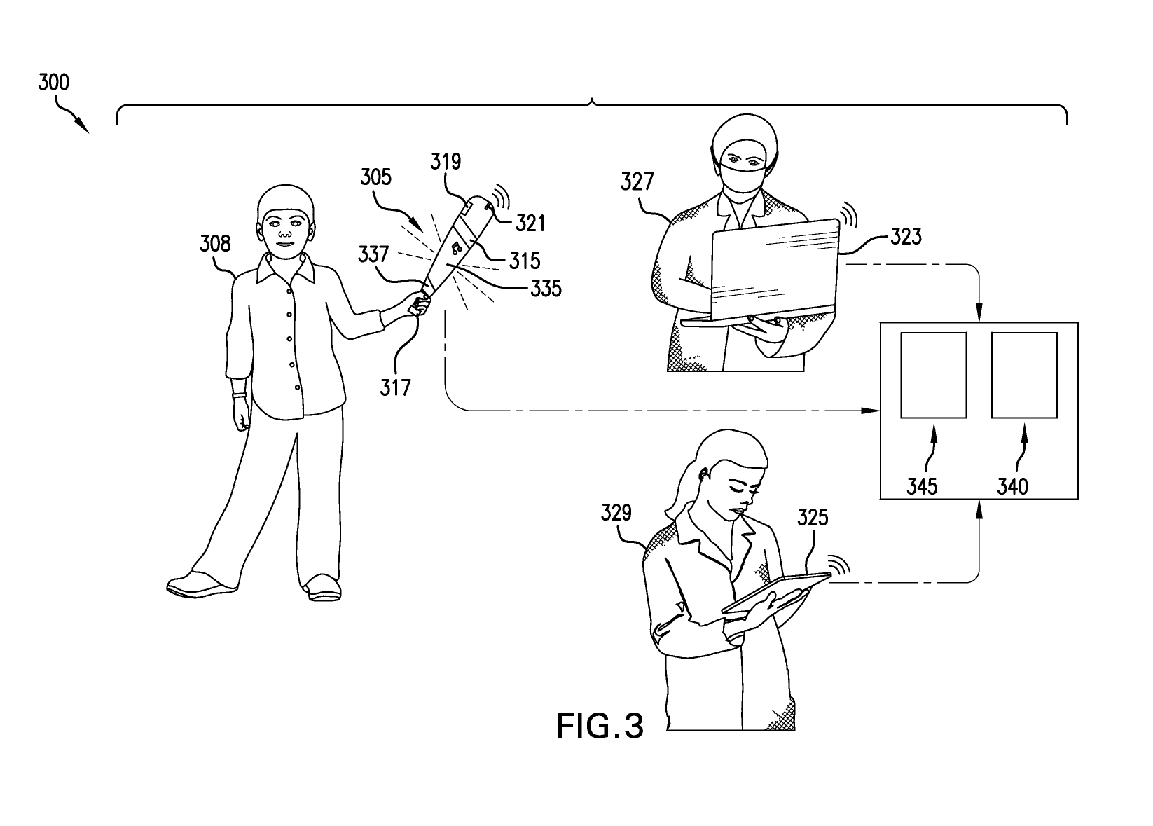

As depicted in FIG. 3, in an embodiment, one or more sensors 317 (thermal, moisture, etc.) are included in the device 205. A processor 315, and the corresponding circuitry in operable communication with the sensors of the device is described in detail in FIG. 4.

In an embodiment, one or more acoustic sensors (e.g., detecting lung sounds from the subject, etc.) 319 are provided in other areas of the toy device 305. In an embodiment, data from the one or more sensors is communicated by way of one or more 321 of a transmitter, receiver, or transceiver configured to communicate with a network, server, remote computing device 323, or mobile device 325 (phone, tablet, etc.) that is accessible to a healthcare worker 327 or parent/guardian or caregiver 329, and the system 300 may interact with an electronic health database 345 or subject's electronic health record(s) 340. In an embodiment, a reward mechanism 335 is included (e.g., flashing lights, musical songs, words of congratulations, etc.) in response to a subject engaging with the device 305 based on the activation of one or more sensors of the device 305. In an embodiment, the device 305 instructs the subject 308 to generally or specifically engage with one or more sensors 317 of the device 305. In an embodiment, the sensors 317 randomly engaged by the subject 308 without prompting are utilized to collect data. In an embodiment, an alert mechanism 337 is included that warns of detection of abnormal data (e.g., biological agents or physiological parameters, etc.) and may include a red light on the tip or another part of the toy, a buzzing noise, siren, or other warning sound.

In an example configuration, as shown in FIG. 4, the system 400 includes a toy device 405 including processing circuitry 416, memory circuitry 417, input/output circuitry 418, user interface (UI) circuitry 419, and sensor circuitry 420 including a at least one of a video camera portion 412, a microphone 439, a moisture sensor 427, a motion sensor 435, or a combination thereof. In an embodiment, the motion sensor 435 includes one or more of an accelerometer (e.g., a post and coil accelerometer), a tilt sensor, or a pressure sensor. In an embodiment, the motion sensor 435 is configured to detect motion. In an embodiment, the accelerator is capable of sensing disposition, acceleration, motion, and/or movement of the medically active toy device. In an embodiment, the acoustic sensor is capable of sensing acoustic energy, such as a noise. In an embodiment, the tilt sensor may be capable of detecting a tilt of the toy device. In an embodiment, the pressure sensor is capable of sensing pressure against the medically active toy device, such as from holding or hugging the toy device. In an embodiment, the moisture sensor 427 is capable of detecting moisture, such as detecting if the subject has put the medically active device 105 or a portion thereof in its mouth, or contacted it with a sweaty hand. In an embodiment, the camera or video recorder 412 is configured to capture still images and/or video and optionally transmit the images to a remote database.

The processor 415 and coordinating circuitry include, for example, processing circuitry 416, memory circuitry 417, input/output circuitry 418, user interface (UI) circuitry 419. Video camera portion 412, and microphone 439 may be coupled together to allow communications therebetween. In an embodiment, the device 405 includes a timer (not shown). Thus, in an embodiment, the medically active toy device 405 can query the subject at a specific time or at specific intervals (e.g., the timer can be programmed to ask every 4 hours if the subject has eaten, or if the subject needs to have its blood glucose tested, etc.).

In an embodiment, the medically active toy device 405 includes a pad of microneedles 457 for sampling the subject (not shown in FIG. 4), for example, for blood glucose or other biological agents. In an embodiment, the microneedles 457 are in operable communication with at least one therapeutic or nutraceutical agent (e.g., insulin) and are utilized for the delivery of the therapeutic or nutraceutical agent.

In an embodiment, an alert 437 is provided to notify the subject or a third party (e.g., a healthcare worker) that at least one set of data is abnormal from a sensor on the device 405.

In an embodiment, the input/output circuitry 418 includes at least one of (421) a receiver, a transmitter, a transceiver, or a combination thereof. In an embodiment, the input/output circuitry 418 is configured to receive and/or provide information relating to interacting with a subject as described herein. For example, the toy device may ask the subject questions that relate to the subject's health (e.g., “Do you have any pain today?” “Are you feeling tired?” “Are you feeling lightheaded or dizzy?”) and/or may observe a subject's behavior by way of the hardware/software described (e.g., tremors, gait, posture, facial expressions, voice, etc.) In an embodiment, observations and/or information related to one or more physiological or biological parameter is transmitted to a third party (e.g., health care worker, caretaker, computing device or system, etc.), and the third party provides an evaluation of the subject based on the transmission. In an embodiment, the third party instructs the toy to specifically question the subject or the third party directly questions the subject through the hardware and/or software of the toy device (e.g., operating the toy remotely).

In an embodiment, the input/output circuitry 418 is configured to communicate with at least one of a computer device 423, or mobile device 425 by way of wireless network or web server, and can include sending and/or receiving at least one of video information, audio information, control information, image information, sensor data, analytical data, location information (global positioning system, assisted global positioning system, etc.). In an embodiment, the input/output circuitry 418 receives and/or sends information via at least one of electromagnetic means (e.g., RF, Wi-fi, Bluetooth, Zigbee, etc.), optical means (e.g., infrared), acoustic means (e.g., speaker, microphone, ultrasonic receiver or transmitter, etc.), or any appropriate combination thereof. In an embodiment, at least one database 445 or the subject's electronic health record 440 are accessed, either wirelessly, or directly as stored in the device 405.

In an embodiment, the memory circuitry 417 includes computer storage media that is volatile (such as dynamic RAM), non-volatile (such a ROM), or a combination thereof. In an embodiment, the system further includes a server 450 that includes additional storage, such as computer storage media (e.g., removable storage or non-removable storage) such as RAM, ROM, EEPROM, tape, flash memory, smart cards, CD-ROM, digital versatile disks (DVD) or devices, or universal serial bus (USB) compatible memory. As described herein, the computer storage medium is an article of manufacture and not a transient signal.

In an embodiment, the device or system further includes at least one input device 460 such as a mouse, pen, keyboard, voice input device, joystick, keypad, thumb pad, or other touch input device, etc., or at least one output device 470 such as a display, speaker, printer, etc.

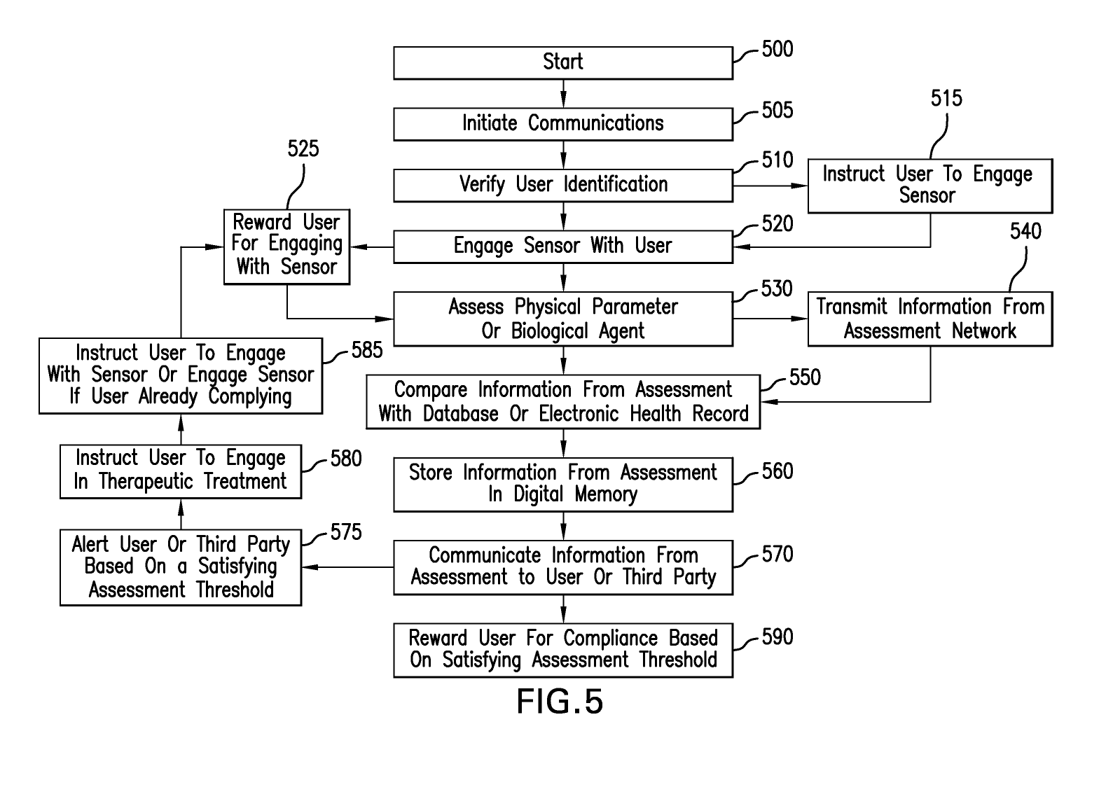

As shown in FIG. 5, a method is started at step 500 by initiating communications at step 505 with a subject (not shown), and optionally verifying subject identification at step 510. Optionally, the device instructs the subject to engage at least one sensor at step 515, and with or without instructions to do so, a sensor is engaged with the subject at step 520. Optionally, a reward is provided to the subject for engaging with the sensor at step 525. One or more physical parameter or biological agent of the subject is assessed at step 530. Optionally, information from an assessment is transmitted to a network or computer system at step 540. Information is compared between the assessment and a database or electronic health record at step 550. Next, information is optionally stored from the assessment in digital memory at step 560. Information is communicated from the assessment to a subject or third party at step 570. Optionally, an alert is given to a subject or third party based on not satisfying an assessment threshold at step 575. Next, the subject is optionally instructed to engage in a therapeutic or nutraceutical treatment at step 580. The therapeutic or nutraceutical treatment may include allowing the device to administer a therapeutic or nutraceutical agent or provide a therapeutic or nutraceutical agent to the subject, or may relate to an external therapeutic or nutraceutical treatment reminder. Next, the subject is optionally instructed to engage with a sensor or the sensor engages if the subject is already complying at step 585. Next, optionally, a reward is provided to the subject for engaging with a sensor at step 525. A reward is provided to the subject for compliance based on satisfying an assessment threshold at step 590, and the method ends at step 595.

As shown in FIG. 6, the device 605 includes at least one housing 625 within an area of the device 605 (for example, in the paw as shown enlarged) that includes a therapeutic or nutraceutical agent reservoir 620, that may also include a label (not shown) for indication of release of the therapeutic or nutraceutical agent from the reservoir 620. In an embodiment, the therapeutic or nutraceutical agent reservoir 620 may be activated to release a therapeutic or nutraceutical agent contained therein in response to a sensor in the toy device, and by way of the pressure sensor 630 that is operably coupled to the swellable hydrogel 640 and puts pressure on the therapeutic or nutraceutical agent reservoir 620 as the hydrogel 640 swells following engagement with a specific biological agent. In an embodiment, the hydrogel 640 has access to a biological fluid by way of a semi-permeable membrane 650. For example, when a subject puts the bear's paw in its mouth, the hydrogel 640 is exposed to the subject's saliva by way of the semi-permeable membrane 650. In an embodiment, the therapeutic or nutraceutical agent in the reservoir 620 may be controllably released by way of a valve 615 operably coupled to a port 610 that leads external to the toy and is configured to contact the subject (e.g., by mouth), depending on the monitoring data generated by detection of one or more biological agents in the subject's saliva. In an embodiment, the therapeutic or nutraceutical agent reservoir 620 is dispensed in response to a sensor (as shown in the Figures) or in response to input by a subject, caregiver, or health care worker (not shown).

### Prophetic Example 1

### A Toy Bear with an Electronic Nose Sensor and Identity Verification Collects and Reports Medical Data to a Child's Caregiver

A toy bear is fabricated with biosensors to determine biological parameters of a child's health as well as visual and audio indications of the child's health. The toy bear interacts with the child using audio and visual cues to command attention and to reward the child. The biosensors and optional additional sensors to measure respiration, cardiopulmonary function, and other physiological parameters are built into the toy bear and connected via microcircuitry to a transmitter which relays the medical data to a caregiver's computer and/or mobile device.

The toy bear incorporates a biosensor to sample and detect chemicals in the breath of the child. For example the toy bear may ask a child with asthma to blow into the bear's ear in order to sample the child's breath. The bear may respond with a chuckle or similar sound when a breath sample is acquired by the biochemical sensor inside the bear's ear. The biosensor designed to detect nitric oxide filtered from the child's breath may include proteins that bind nitric oxide and optics to measure that binding. For example, portable nitric oxide analyzers available from Aerocrine AB (Solna, Sweden) and described in U.S. Pat. No. 8,206,311 (which is incorporated herein by reference) measure fractional nitric oxide levels in exhaled breath in concentrations ranging from 5 to 300 parts per billion of exhaled breath. Alternatively or in addition, the toy bear includes an electronic nose sensor positioned in the bear's nose, and the child is instructed to blow into the nose. The electronic nose sensor includes an array of quartz microbalance gas sensors coated with molecular films of metalloporphyrins, which detect the amount of nitric oxide absorbed in the film through changes of resonant frequency proportional to the absorbed mass. Programming and circuitry analyze the frequency shifts. The use of an electronic nose to test nitric oxide in breath is described in Montuschi et al., Chest 137(4):790-796; 2010, which is incorporated herein by reference. Periodic measurements and analyses of nitric oxide levels are transmitted to a caregiver's computer or mobile device to monitor the child's asthma and to guide therapy (see e.g., Smith et al., N. Engl. J. Med. 352:2163-73, 2005 which is incorporated herein by reference).

In addition, a biosensor comprising a commercially available electronic nose (from Smiths Detection, Edgewood, Md.) that is based on an array of 32 conducting polymer sensors, may be incorporated into the toy to measure volatile organic chemicals in exhaled breath that represent disease markers. For example, biomarkers of asthma, bacterial infection, and upper respiratory tract infections may be detected by the electronic nose (see e.g., Wilson and Baietto, Sensors 11:1105-1176, 2011, which is incorporated herein by reference). In addition, analysis of exhaled breath condensate may be used to detect biomarkers including cytokines, prostaglandins and leukotrienes. Biomarkers related to airway disease that are found in exhaled breath condensate are described (see e.g., Kharitonov and Barnes, Am J Respir Crit Care Med 163:1693-1722, 2001; Kharitonov and Barnes, Chest 130:1541-1546, 2006, and Robroeks et al., Clin. Exp. Allergy 37:1303-1311, 2007, each of which is incorporated herein by reference).

Data on the biomarkers detected by the one or more biosensors are transmitted wirelessly to a caregiver's computer or mobile device and analyzed by system software. Computer programs to analyze the biomarker data are described (see e.g., International Publication No. WO 2005/100235, Ibid.) and can be adapted to this embodiment.

The toy bear includes sensors to monitor the child's electrocardiogram (ECG) and blood oxygen content. For example, nonconductive electrodynamic sensors are built into the bear's paws to detect ECG signals when the child holds the bear's hands. Audio prompts are given by the bear to encourage the child to hold each of the bear's two upper paws, and ECG signals are captured automatically when the sensor probes are in contact with the child's hands or within approximately 2 mm of the child's hands. Electrodynamic sensors that collect ECG signals using nonconductive probes or noncontact probes are described (see e.g., U.S. Patent App. Pub. No. 2006/0058694, which is incorporated herein by reference). Successful capture of ECG signals is rewarded by sounds and lights from the toy bear. For example, a favorite song may play and the bear's heart (outlined by LEDs) may glow. ECG data collected by the toy bear is transmitted to a caregiver's mobile device or computer. The mobile device and/or computer sounds an alert if the ECG data is abnormal or incomplete (e.g., ECG signals not transmitted at a scheduled time). A pulse oximeter is also incorporated in the toy bear to monitor heart rate and oxygen saturation of hemoglobin (Hb). The oximeter sensor is incorporated in one of the bear's paws, which has a grasping mechanism such as a spring or Velcro. The sensor detects transmission of infrared light (approximately 940 nm wavelength) and red visible light (approximately 660 nm) through the child's finger when the bear's paw grasps the child's finger. A microprocessor calculates the Hb oxygen saturation and pulse rate based on the ratio of transmitted infrared and red light. Pulse oximetry sensors and sensor components to determine blood oxygenation are well known and are available from suppliers including Advanced Photonix, Inc., Camarillo, Calif., and can be adapted for use with the present embodiment. Successful determination of the child's pulse and blood oxygenation is rewarded with lights and sounds as described above, i.e., music and flashing lights. The child's pulse and blood oxygen levels are transmitted wirelessly to the caregiver's computer and/or mobile device where the data are analyzed. An alert is sounded if the oxygen saturation levels are abnormal (e.g., low oxygen saturation may be associated with asthma or other respiratory disease) or if the pulse is abnormal.

The toy bear also includes nonconductive electrodynamic sensors to collect electrical signals from the child's brain, for example, electroencephalogram (EEG) signals. The toy bear audio function prompts the child to place the toy bear's stomach on the top of his or her head in order to place probes from electrodynamic sensors in physical contact with the head of the child. Methods and electrodynamic sensors to collect EEG signals are described (see e.g., U.S. Patent App. Pub. No. 2006/0058694, Ibid.). Successful collection of EEG traces, requiring approximately 10-30 seconds, is rewarded with a song and flashing lights. EEG signals are sent to microprocessors in the toy bear prior to wireless transmission to the caregiver's computer or mobile device. EEG signals may indicate epileptic seizures, or foreshadow epileptic seizures. For example an EEG signal pattern may indicate an impending seizure. Algorithms to identify predictive EEG patterns are described (see e.g., Williamson et al., Epilepsy and Behavior 25:230-238, 2012, which is incorporated herein by reference). The toy bear may warn the subject (e.g., child) that a seizure may be imminent and also alert the caretaker that a seizure may occur.

The toy bear also includes a video camera and image processors to identify the child, to facilitate interaction with the toy bear, and to monitor the child. For example a video camera may be incorporated in the toy bear's face and actuated when the child's presence is detected by means of infrared sensors. Video images are processed by face recognition software in the toy bear's computer to identify the child and to initiate audio communication. For example, the toy bear may greet the child and ask for a hug. Video cameras, face recognition software and infrared sensors are described (see e.g., U.S. Patent App. Pub. No. 2009/0055019, which is incorporated herein by reference). The video camera may be programmed to monitor the child continuously for longer periods of time. For example, the child may be monitored overnight to capture video and audio signals. Image processing and audio signal processing by the toy's computer system is programmed to alert the child's caretaker if regular breathing stops, or coughing and wheezing occur (e.g., asthma, sudden infant death syndrome, or respiratory distress). In addition, the toy bear can calm the child with soothing music or the mother's voice if breathing or movement irregularities are detected.

### Prophetic Example 2

### A Toy Doll for Collection and Analysis of Biological Fluids and Administration of Medicaments

A toy doll is fabricated to sample biological fluids, detect biological agents and administer medicaments to a child. The toy identifies the child and establishes wireless contact with the child's electronic health record (EHR) to receive and transmit personal and health information. Prescribed tests and medications are programmed by a caregiver and verified on the EHR.

The toy doll identifies the child and accesses the child's EHR using wireless communication. For example the child may wear an RFID bracelet which is recognized by an RFID reader on the doll. The bracelet is constructed with an RFID tag that contains antennas and circuitry to receive and transmit radio frequency signals that identify the child wearing the RFID bracelet. Methods and circuitry to construct RFID tags are described (see e.g., U.S. Pat. No. 7,479,886 and U.S. Pat. No. 6,693,513; each of which is incorporated herein by reference). An RFID tag with an antenna for harvesting power at UHF frequencies and transmitting backscatter signals to the RFID reader may be constructed with circuitry to send an identification signal, the time and date, and signals from additional sensors (see e.g., Sample et al., IEEE Trans. Instr. Meas. 57:2608-2615, 2008 which is incorporated herein by reference). The RFID reader in the doll receives RFID signals and transmits to a computer or mobile device signals that identify the child and allow access to the corresponding EHR. The toy doll may also receive signals from a caregiver, via a remote computer or mobile device that activate the doll for sample collection and biological agent analysis.

The caregiver may initiate sample collection by the toy doll at a chosen time, or the toy doll may be programmed to automatically collect a biological sample at a given time each day, every 4 hours or after meals. For example a child with diabetes requires regularly scheduled blood sampling to monitor blood glucose, hemoglobin 1Ac, and insulin C-peptide levels. To obtain a blood sample the toy doll asks the child to burp it by patting a spot on its back where the toy doll has a patch of microneedles. The doll burps or makes chortling sounds when a blood sample has been obtained. The microneedles collect approximately 50 microliters of blood, which is aspirated by a microfluidic system in the toy doll for determination of biological agents including glucose, HbA1c, and insulin. All or part of the microfluidics and microneedles are removable from the bear for access and portions, including the microneedles and cartridge, are disposable. A point of care, battery-powered microfluidic system for analysis of whole blood is described by Maleki et al., Proc. SPIE 8251, Microfluidics, BioMEMS, and Medical Microsystems X, 82510C, 2012; doi:10.1117/12.909051, each of which is incorporated herein by reference. The data on blood biological agents is transmitted by the toy doll system to a remote computer or mobile device and entered into the child's EHR. A caregiver may be alerted if any of the biological agent levels are abnormal or require repeat analysis. In addition the toy doll is equipped to collect and analyze saliva samples from the child. The doll hand comprises an ice cream cone, and the doll asks the child to lick the cone, where a semipermeable membrane covers a channel for collection of saliva; the cone may include flavoring. For example, devices to collect salivary fluid are available and may be adapted for use in this embodiment. (See e.g., U.S. Pat. No. 6,022,326, which is incorporated herein by reference). The doll may giggle if the sample is obtained, or ask for another lick if more saliva is needed. The saliva sample is analyzed using a microfluidic system incorporated in the toy doll.

Biological agents that may include proteins, metabolites, pharmaceuticals and microbes are detected in saliva. For example, salivary fluid may be used to determine immunization to, or infection with, measles virus, mumps virus and rubella virus. Anti-viral antibodies (IgG) in salivary fluid have been identified as having respective sensitivities and specificities of 97% and 100% for measles, 94% and 94% for mumps, and 98% and 98% for rubella, in comparison with detection of serum antibodies for these viruses. See, for example, Thieme et al., “Determination of measles, mumps and rubella immunization status by using oral fluid samples,” JAMA 272:219-221 (1994), which is incorporated herein by reference. Microfluidic systems to determine antibodies, nucleic acids, small molecules and other bio-biological agents are described (see e.g., Cho et al., “Recent advances in microfluidic technologies for biochemistry and molecular biology,” BMB Reports 44:705-711, 2011, which is incorporated herein by reference). The biosensor might instead, or as a component of the microfluidics, utilize a quartz crystal microbalance biosensor for monitoring antibodies that carries as its binding molecule an immobilized anti-Ig antibody (e.g. an anti-idiotypic antibody) or viral antigen. (See, e.g., Tajima et al., Abstract; Analytica Chimica Acta 365(165):147PERL; 1998, which is incorporated herein by reference).

Portions of the toy can optionally include modular portions for replaceable biosensors. For example, the ice cream cone opens for removal of the immunoglobulin biosensor and replacement with a biosensor for measuring hormone levels. For example a child on the autism spectrum is monitored occasionally for antibody levels but is monitored frequently for levels of cortisol in saliva for indications of stress and anxiety. For example, the ice cream cone is fitted with a mu-electrode (IDmE) based impedimetric cortisol biosensor carrying a functionalized antibody as a binding molecule that monitors the cortisol levels in saliva (see, e.g., Arya et al., Analyst 135:1941-1946, 2010, which is incorporated herein by reference).

Electronic signals from the biosensors are processed by the circuitry in the doll system, and the biological agent data is transmitted wirelessly to the caregiver's computer or mobile device. For example, the bioanalytic data, the child's name, and the date and time are also transmitted to the child's EHR. The toy doll may be programmed to alert the child or the caregiver or both if biological agent levels require immediate attention. For example, a high blood glucose value may trigger an alert to the child and the caregiver that an insulin injection is required.

The toy doll includes components to administer medicaments to the subject, i.e. the child, and the doll interacts with the child before, during, and after drug administration. For example, if the doll detects elevated blood glucose levels, e.g., greater than 230 mg/dL, the child may be alerted by the doll that it is time to “hold hands with the doll”. The audio prompt tells the child to hold the doll's hand where a microneedle patch is located. Holding the doll's hand allows the doll to inject insulin subcutaneously in the child's hand through the microneedle patch. Microneedles for transdermal injection of insulin and other medicaments are known (see e.g., McAllister et al., Proc. Natl. Acad. Sci. USA 100:13755-13760, 2003, which is incorporated herein by reference). The doll encourages the child to hold its hand until the insulin has been administered, and upon completion of the injection the doll praises the child and plays a favorite song as a reward. Microneedle patches to administer insulin may be discarded and replaced by the child's caretaker. Also the insulin injection is recorded by the toy control circuitry and data on the injection is transmitted to a remote computer and entered into the child's EHR. The child's caretaker is also alerted that insulin has been administered via wireless transmission to a mobile device.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A physically contactable toy device, comprising:
at least one physically contactable housing unit;
at least one computer processor;
at least one biosensor;
and at least one at least one means to identify the subject.

2. (canceled)

3. (canceled)

4. The physically contactable toy device of claim 1, wherein the biosensor includes at least one detector for detecting one or more biological agents from sweat, saliva, mucus, blood, or breath.

5. The physically contactable toy device of claim 4, wherein the one or more biological agents include one or more pathogens.

6. (canceled)

7. (canceled)

8. (canceled)

9. (canceled)

10. (canceled)

11. (canceled)

12. The physically contactable toy device of claim 1, further including at least one of an acoustic sensor, pulse oximeter, electrode, thermal sensor, or moisture sensor.

13. The physically contactable toy device of claim 12, wherein the acoustic sensor is configured to sense wheezing in the subject.

14. The physically contactable toy device of claim 1, further including at least one nonconductive, remote sensor.

15. The physically contactable toy device of claim 14, wherein the at least one nonconductive, remote sensor includes at least one electroencephalography (EEG), electrocardiography (ECG), electromyography (EMG), or sensor.

16. (canceled)

17. The physically contactable toy device of claim 1, further including at least one of a video camera, pressure sensor, motion sensor, RFID reader, optical scanner, laser reader, or clock.

18. The physically contactable toy device of claim 17, wherein the motion sensor includes at least one of a tilt sensor, pressure sensor, or an accelerometer.

19. The physically contactable toy device of claim 1, further including signal output component operably coupled to the at least one biosensor and configured to generate at least one signal in response to activation of the at least one biosensor.

20. The physically contactable toy device of claim 1, wherein the at least one means to identify the subject includes at least one of an RFID scanner, optical scanner, sensor, imager, biometric data scanner, or input device.

21. (canceled)

22. (canceled)

23. The physically contactable toy device of claim 1, wherein means for identifying the subject includes at least two different means.

24. The physically contactable toy device of claim 1, further including at least one transmitter.

25. The physically contactable toy device of claim 1, further including at least one receiver.

26. The physically contactable toy device of claim 1, further including at least one means for generating an output to a subject, another computer, or a healthcare worker.

27. (canceled)

28. (canceled)

29. (canceled)

30. (canceled)

31. (canceled)

32. (canceled)

33. (canceled)

34. (canceled)

35. (canceled)

36. The physically contactable toy device of claim 1, further including means for collecting at least one biological sample from a subject.

37. (canceled)

38. The physically contactable toy device of claim 1, further including a user control interface.

39. (canceled)

40. The physically contactable toy device of claim 1, further including an operator interface.

41. (canceled)

42. (canceled)

43. (canceled)

44. The physically contactable toy device of claim 1, wherein one or more of the at least one sensors is enmeshed in fabric of the device.

45. The physically contactable toy device of claim 1, further including at least one means for containing and dispensing at least one therapeutic or nutraceutical agent.

46. (canceled)

47. (canceled)

48. (canceled)

49. (canceled)

50. (canceled)

51. (canceled)

52. The physically contactable toy device of claim 1, wherein the device is personalized for a specific subject, based on the subject's health status or suspected health status.

53. The physically contactable toy device of claim 1, further including at least one memory storage device in communication with the at least one computer processor.

54. (canceled)

55. The physically contactable toy device of claim 1, further including at least one signal receiver in communication with at least one remote database.

56. The physically contactable toy device of claim 1, further including at least one sampling component.

57. (canceled)

58. (canceled)

59. The physically contactable toy device of claim 1, further including one or more of a microfluidics or nanofluidics chip, thermocycler, immunoassay component, or gel or liquid microcolumn.

60. (canceled)

61. (canceled)

62. (canceled)

63. (canceled)

64. A system comprising:
at least one computing system operably coupled to a physically contactable toy device including,
at least one computer processor;
at least one housing unit;
at least one biosensor;
and at least one at least one means to identify the subject.

65. The system of claim 64, further including at least one medical database stored in the toy device or stored remotely.

66. The system of claim 65, wherein the at least one medical database includes at least one of a specific subject's electronic health records or specific medical information tailored to a specific subject.

67. The system of claim 64, wherein the physically contactable device is handheld.

68. The system of claim 64, wherein the physically contactable toy device further includes processing circuitry.

69. The system of claim 64, wherein the physically contactable toy device further includes memory circuitry.

70. The system of claim 64, wherein the physically contactable toy device further includes input/output circuitry.

71. The system of claim 64, wherein the physically contactable toy device further includes user interface (UI) circuitry.

72. The system of claim 64, wherein the physically contactable toy device further includes sensor circuitry.

73. The system of claim 64, wherein the physically contactable toy device further includes at least one of a video camera, microphone, moisture sensor, motion sensor, or a combination thereof.

74. The system of claim 73, wherein the motion sensor includes one or more of an accelerometer, a tilt sensor, or a pressure sensor.

75. The system of claim 64, wherein the physically contactable toy device includes at least one of a transmitter, receiver, or transceiver.

76. The system of claim 64, further including at least one audio or video recorder.

77. The system of claim 64, further including at least one signal output component operably coupled to the at least one sensor and configured to generate at least one signal in response to activation of the at least one biosensor.

78.-90. (canceled)
